(19) United States
(12) Patent Application Publication
(10) Pub. No.: US 2010/0113880 A1
(43) Pub. Date: May 6, 2010

(54) Gastrostomy-jejunostomy tube apparatus and method for endoscopically placing same within a patient

(76) Inventor: Charles W. Page, Nacogdoches, TX (US)

Correspondence Address: DAVID ODELL SIMMONS, 7637 PARKVIEW CIRCLE, AUSTIN, TX, 78731, US

(21) Appl. No.: 12/290,963

(22) Filed: Nov. 5, 2008

(51) Int. Cl. A61B 1/018

(52) U.S. Cl. 600/153

(57) ABSTRACT

A percutaneous endoscopic gastrostomy-jejunostomy (G-J) tube apparatus has a percutaneous endoscopic gastrostomy-jejunostomy (PEG-J) tube unit and a jejunostomy (J-tube). The PEG-J tube unit includes two interconnected tubes extending in a side-by-side orientation with respect to each other and a posterior bumper encompassing both of the interconnected tubes adjacent a first end portion thereof. Each one of the interconnected tubes includes a passage extending therethrough between opposing end faces thereof. A first one of the interconnected tubes is configured for functioning as a gastrostomy tube (G-tube) and a second one of the interconnected tubes is configured for functioning as a jejunostomy tube (J-tube) introducer. The J-tube is slideably inserted into a passage of the J-tube introducer. The J-tube includes a plug portion integral therewith and disposed within the passage of the J-tube introducer for providing a fluid-resistant seal between the J-tube and the J-tube introducer.

### FIELD OF THE DISCLOSURE

The disclosures made herein relate generally to gastrostomy feeding tube apparatuses and, more particularly, to gastrostomy feeding tube apparatuses that are configured for allowing simultaneous delivery of flowable substances (e.g., nutrients and/or medications) above and below the Pylorus portion of the gastric tract.

### BACKGROUND

A gastrojejunal feeding tube apparatus helps to decrease the probability of aspiration in a patient. Aspiration is the condition where fluid in the stomach moves back into the esophagus (i.e., gastro-esphageal reflux), up the esophagus and then down the trachea and lungs. As a result, gastro-esophageal reflux can lead to aspiration-induced pneumonia.

There are several conditions that predispose a patient to aspiration. One such condition is a stroke, which creates swallowing problems (e.g., Esphageal Dysmotility) and alters normal progression of food down the esophagus. Another such condition is Gastro-Esphageal Reflux Disease (GERD) in which the lower esophageal sphincter (LES) is loose and allows gastric contents to reflux (i.e., move in the wrong direction). Another such condition is Gastroparesis, which is a condition common in diabetics where food/fluids build up in the stomach because the stomach does not properly move contents thereof down into the Duodenum. Still another such condition is Paralytic Ileus, which is a condition that develops from surgery, trauma, medication or the like and results in the bowel becoming inactive such that it does not push through contents thereof in a normal manner.

To mitigate issues associated with aspiration resulting from gastro-esophageal reflux, a Gastrostomy-Jejunostomy (G-J) feeding tube apparatus can be utilized. The G-J tube apparatus enables tube feeds to be administered at a location below the pyloric valve (e.g., within the duodenum) via a jejunostomy tube and aspiration of the stomach via a gastrostomy tube. In this manner, the probability of aspiration resulting from reflux is greatly reduced while still allowing for gastrojejunal feeding.

However, for any number of reasons, currently known G-J tube apparatuses cannot be placed endoscopically during an initial gastrostomy placement procedure. As such with current technology/product offerings, it is not possible to simultaneously place both a gastrostomy tube and a jejunostomy tube. Accordingly, common practice is to install a Percutaneous Endoscopic Gastrostomy (PEG) tube apparatus and then, after the gastrostomy site has matured (i.e., stomach wall typically adheres to the abdomen wall at the gastrostomy site within about a month), the PEG tube apparatus is removed and a G-J tube apparatus (i.e., gastrostomy and jejunostomy tubes are unitarily formed) is placed back into the gastrostomy site and a jejunostomy tube of the G-J tube apparatus is endoscopically placed below the pyloric valve.

Therefore, a PEG-J tube apparatus that overcomes the drawbacks associated with conventional percutaneous endoscopic apparatuses and associated placement procedures would be advantageous, desirable and useful.

### SUMMARY OF THE DISCLOSURE

Embodiments of the present invention enable a percutaneous endoscopic gastrostomy-jejunostomy (PEG-J) tube apparatus to be placed in a patient during a single endoscope procedure. In doing so, the need to wait the customary period of time for the placement of a gastrostomy or jejunostomy tube to mature prior to replacing such gastrostomy tube or jejunostomy tube with a gastrostomy-jejunostomy tube apparatus is eliminated. Accordingly, embodiments of the present invention advantageously overcome one or more shortcomings associated with conventional approaches for placing a gastrostomy-jejunostomy tube apparatus.

In one embodiment of the present invention, a method for providing a jejunostomy tube and a gastrostomy tube within a body of a patient during a single endoscopic procedure comprises a plurality of steps. A step is provided for endoscopically placing a gastrostomy-jejunostomy (G-J) tube apparatus through abdominal and stomach walls of the patient such that a first end portion of the G-J tube apparatus is exposed within the gastric lumen of the stomach and a second end portion of the G-J tube apparatus is exposed outside of the patient's body. The G-J tube apparatus includes provisions configured for compressing together portions of the walls encompassing the G-J tube apparatus. The G-J tube apparatus includes two interconnected tubes extending in a side-by-side orientation with respect to each other. A first one of the interconnected tubes is configured for functioning as a gastrostomy tube (G-tube) and a second one of the interconnected tubes is configured for functioning as a jejunostomy tube (J-tube) introducer. A step is provided for inserting a first end portion of a J-tube into a passage of the J-tube introducer from an end portion thereof located outside of the patient's body. Such inserting of the J-tube is performed in a manner that causes the first end portion of the J-tube to become exposed within the gastric lumen of the stomach. After or in concert with performing such inserting of the J-tube, a step is performed for endoscopically placing the first end portion of the J-tube through a pyloric valve of the patient.

In another embodiment of the present invention, a gastrostomy-jejunostomy (G-J) tube apparatus comprises a gastrostomy-jejunostomy (G-J) tube unit and a jejunostomy (J-tube). The G-J tube unit includes two interconnected tubes extending in a side-by-side orientation with respect to each other and a posterior bumper encompassing both of the interconnected tubes adjacent a first end portion thereof. Each one of the interconnected-tubes includes a passage extending therethrough between opposing end faces thereof. A first one of the interconnected tubes is configured for functioning as a gastrostomy tube (G-tube) and a second one of the interconnected tubes is configured for functioning as a jejunostomy tube (J-tube) introducer. The J-tube is slideably inserted into a passage of the J-tube introducer. The J-tube includes a plug portion attached thereto and disposed within the passage of the J-tube introducer for providing a fluid-resistant seal between the J-tube and the J-tube introducer.

In another embodiment of the present invention, a system of components configured for performing placement and maintenance of a gastrostomy-jejunostomy (G-J) tube apparatus comprises a gastrostomy-jejunostomy (G-J) tube unit, an anterior bumper and ajejunostomy tube (J-tube. The gastrostomy-jejunostomy (G-J) tube unit includes two interconnected tubes extending in a side-by-side orientation with respect to each other and a posterior bumper encompassing both of the interconnected tubes adjacent a first end portion of the G-J tube unit. A first one of the interconnected tubes is configured for functioning as a gastrostomy tube (G-tube) and a second one of the interconnected tubes is configured for functioning as a jejunostomy tube (J-tube) introducer. The anterior bumper has a passage extending through a central portion thereof. The passage is configured for having the interconnected tubes extending therethrough. The J-tube includes an elongated portion configured for being slideably inserted through a passage of the J-tube introducer and a plug portion attached to the elongated portion thereof. The plug portion is configured for being engaged within a passage of the J-tube introducer for providing a fluid-resistant seal between the J-tube and the J-tube introducer.

These and other objects, embodiments, advantages and/or distinctions of the present invention will become readily apparent upon further review of the following specification, associated drawings and appended claims.

### BRIEF DESCRIPTION OF THE DRAWINGS

FIG. 1 is an illustrative view showing a percutaneous endoscopic gastrostomy-jejunostomy (PEG-J) tube apparatus configured in accordance with an embodiment of the present invention and endoscopically placed within a patient.

FIG. 2 is a perspective view showing a PEG-J tube unit of the PEG-J tube apparatus of FIG. 1, shown prior to being endoscopically placed within a patient.

FIG. 3 is a cross-sectional view taken along the line 3-3 in FIG. 2.

FIG. 4 is a fragmentary cross-sectional view showing an interconnected tube portion and J-tube of the PEG-J tube apparatus of FIG. 1, wherein the interconnected tubes are fully connected to each other and the jejunostomy tube (J-tube) in a partially inserted orientation.

FIG. 5 is a fragmentary cross-sectional view showing an interconnected tube portion and J-tube of the PEG-J tube apparatus of FIG. 1, wherein the interconnected tubes are partially separated from each other.

### DETAILED DESCRIPTION OF THE DRAWING FIGURES

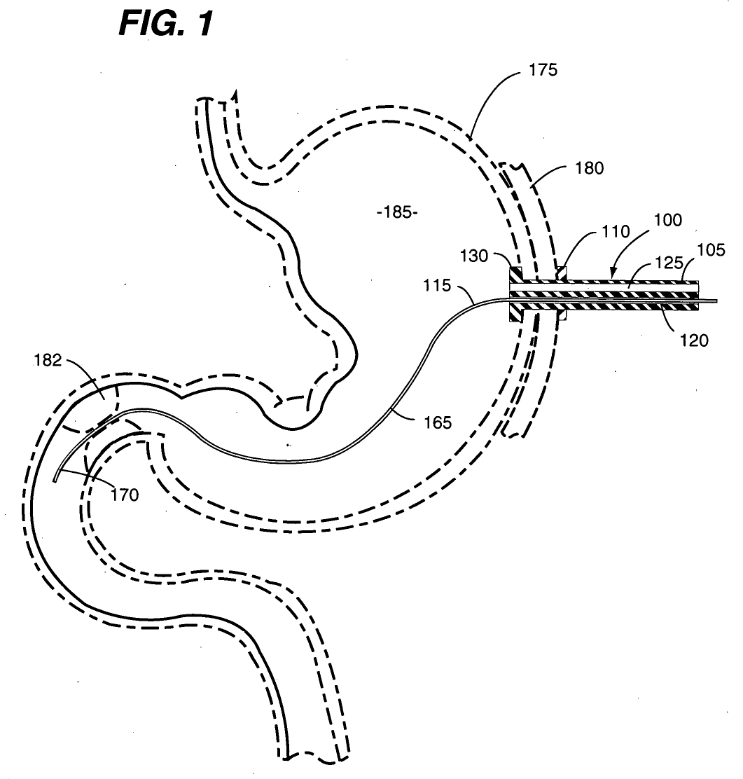

FIGS. 1-5 each show various aspects of an embodiment of a percutaneous endoscopic gastrostomy-jejunostomy (PEG-J) tube apparatus 100 configured in accordance with the present invention. Advantageously, the PEG-J tube apparatus 100 enables a percutaneous endoscopic gastrostomy-jejunostomy (PEG-J) tube apparatus to be placed in a patient during a single endoscope procedure. In doing so, the need to wait the customary period of time after surgical placement of a gastrostomy or jejunostomy tube for a resulting interface between the stomach and abdominal walls to mature (i.e., seal together) prior to replacing such gastrostomy tube or jejunostomy tube with a gastrostomy-jejunostomy (G-J) tube apparatus is eliminated. Accordingly, the PEG-J tube apparatus 100 advantageously overcomes one or more shortcomings associated with conventional approaches for placing gastrostomy-jejunostomy tube apparatuses.

The PEG-J tube apparatus 100 includes a PEG-J tube unit 105, an anterior bumper 110 and a J-tube 115. The PEG-J tube unit 105 includes two interconnected tubes 120, 125 extending in a side-by-side orientation with respect to each other, a posterior bumper 130 encompassing both of the interconnected tubes 120, 125 adjacent a first end portion 135 of the PEG-J tube unit 105, and an tool engagable structure 140 integral with a second end portion 145 of the PEG-J tube unit 105. Each one of the interconnected tubes 120, 125 includes a respective passage 120′, 125′ that extends through a face of a first end 150 and are terminated by the tool engagable structure 140. The anterior bumper 110 includes an opening therein that is configured for having the interconnected tubes 120, 125 extend therethrough. Preferably, but not necessarily, the opening is configured to provide an interference fit between the anterior bumper 110 and the interconnected tubes 120, 125 such that the anterior bumper 110 will maintain a prescribed position on the interconnected tubes 120, 125 without human intervention (e.g., via friction). For example, the opening can be substantially the same shape and size as the exterior cross sectional shape of the interconnected tubes 120,125 (e.g., oval). Alternatively, additional means for securing the anterior bumper 110 in a fixed position with respect to the interconnected tubes 120, 125 can be provided.

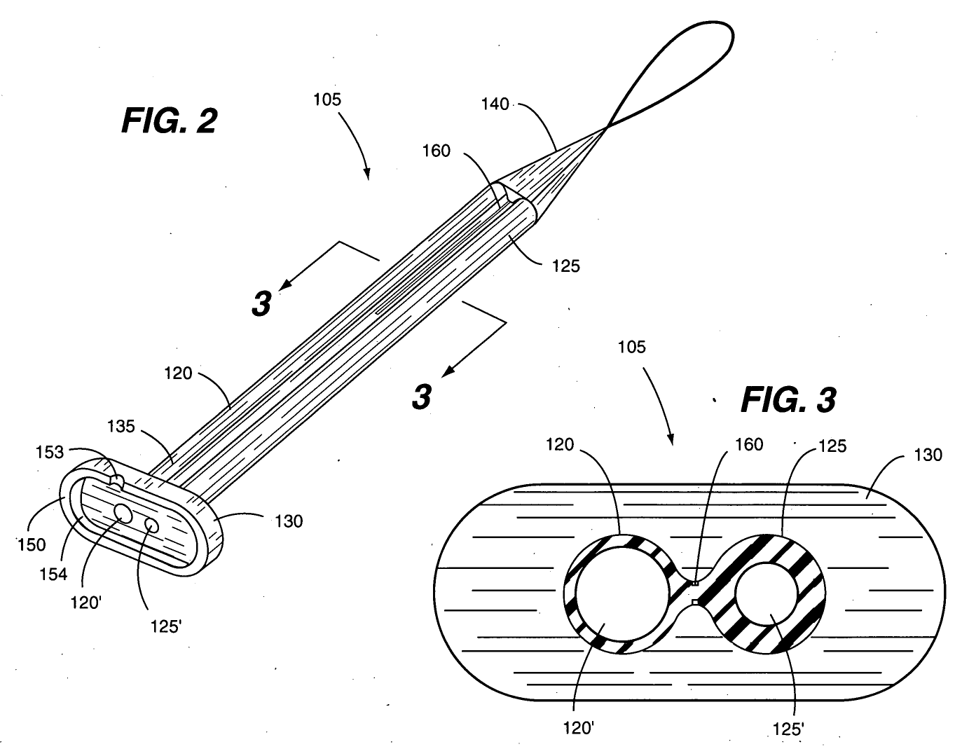

The posterior bumper 130 can be unitarily formed with the interconnected tubes 120, 125 or a discrete component attached to the interconnected tubes 120, 125 (e.g., slideably engaged therewith abutting an integral flange of the interconnected tubes 120, 125). As shown in FIG. 2, a face of the posterior bumper 130 that faces into the gastric lumen includes a recessed portion 153 and at least one bleed passage 154. Jointly, the recessed portion 153 and at least one bleed-passage 154 aid in limiting the potential for opposing wall of other structure in the gastric lumen from being fully drawn against the posterior bumper when suction is applied to the G-tube 120 for performing aspiration.

A first one of the interconnected tubes is configured for functioning as a gastrostomy tube (i.e., G-tube 120) and a second one of the interconnected tubes is configured for functioning as a jejunostomy tube introducer (i.e., J-tube introducer 125). In one embodiment (as shown), the passage 120′ of the G-tube 120 has a different inside diameter (i.e., inside dimension) than does the passage 125′ of the J-tube introducer 125. Alternatively, the passage 120′ of the G-tube 120 can be the same as that of the passage 125′ of the J-tube introducer 125. Functionally, the G-tube 120 is configured for having a fluid flow therethrough.

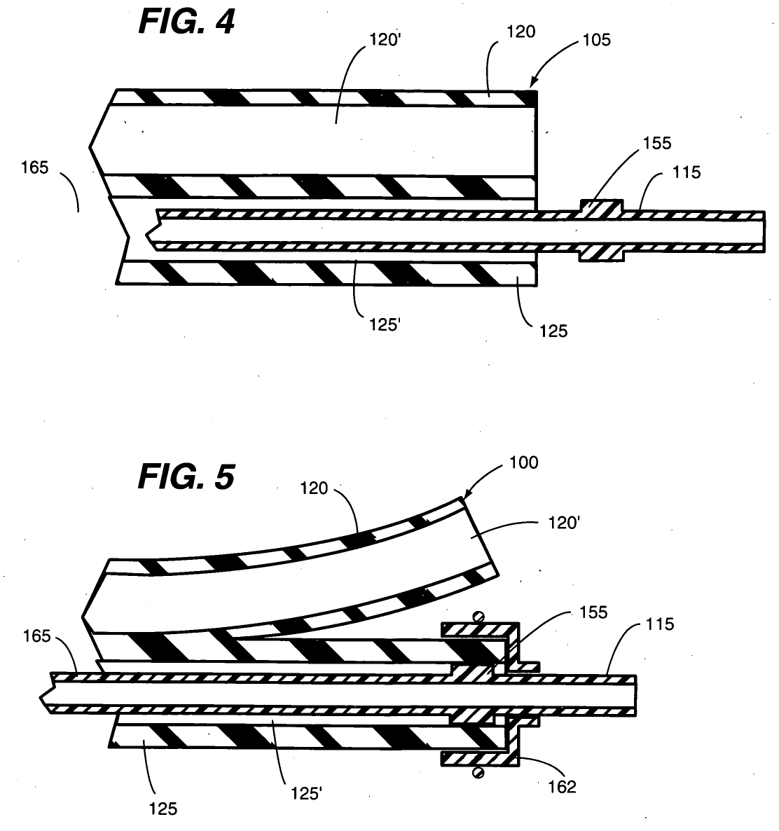

As best shown in FIGS. 4 and 5, the J-tube 115 includes a plug 155. The plug 155 is a seal providing means (e.g., a seal), which can be a unitarily formed portion of the J-tube 115 or a discrete component. The plug 155 is configured for being disposed within the passage 125′ of the J-tube introducer 125 for providing a fluid-resistant seal between the J-tube 115 and the J-tube introducer 125. In the case where the passage 125′ of the J-tube introducer 125 has a larger inside diameter (i.e., inside dimension) than the outside diameter (i.e., outside dimension) of the J-tube 115, such seal is intended to preclude the unrestricted flow of fluids and other flowable matter in the space between the J-tube introducer 125 and the J-tube 115.

Preferably, but not necessarily, the G-tube 120 and J-tube introducer 125 (i.e., the interconnected tubes) are connected to each other in a manner allowing them to be manually peeled apart from each other generally along a prescribed interface. As shown in FIGS. 2 and 3, one or more grooves 160 can be provided between the G-tube 120 and J-tube introducer 125 for providing a preferential path along which such peeling takes place. FIG. 5 shows the G-tube 120 and J-tube introducer 125 in the peeled apart (i.e., separated) configuration. Optionally, a seal 162 can be secured of the separated end of the J-tube introducer 125 by fastening means such as, for example, a twisted wire or plastic cable tie. In such an arrangement, the plug 155 and the seal 162 jointly provide a fluid-resistant seal between the J-tube 115 and the J-tube introducer 125. It is also disclosed herein that the seal 162 can be configured to be engaged over the interconnected tubes 120, 125 when they are in the non-separated configuration.

Referring now to FIG. 1, the passage 125′ of the J-tube introducer 125 is configured for having an elongated portion 165 of the J-tube 115 slideably inserted into the passage 125′. The elongated portion 165 has a substantially uniform outside diameter (i.e., outside dimension) that allows an entire length of the elongated portion 165 to be slideably inserted into the passage 125′ of the J-tube introducer 125. In particular, a tip region 170 of the elongated portion 165 has an outside diameter that is substantially the same as the remainder of the elongated portion 165.

Still referring, to FIG. 1, in use, the PEG-J tube unit 105 is placed such that the interconnected tubes 120, 125 extend through the stomach wall 175 and abdominal wall 180 of a patient with the posterior bumper 130 engaged with the stomach wall 175 (i.e., the gastric mucosa) inside the gastric lumen 185. The anterior bumper 110 is engaged over the interconnected tubes 120, 125 sufficiently to engage the abdominal wall 180 to cause the stomach wall 175 and abdominal wall 180 to become compressed together between the anterior bumper 110 and the posterior bumper 130. The J-tube 115 extends through the passage 125′ of the j-tube introducer 125. The plug 155 of the J-tube 115 is engaged within the passage 125′ of the j-tube introducer 125 to form a seal therebetween. The tip region 170 of the J-tube 115 is situated past the pyloric valve 182 of the patient. In this manner, the J-tube 115 can be used for providing tube feeds below the pyloric valve and the gastrostomy tube 120 of the PEG-J tube unit 105 can be used for carrying out aspiration of the stomach.

Advantageously, a PEG-J tube apparatus configured in accordance with the present invention can be placed using endoscopic techniques. The benefit is that the J-tube of such a PEG-J tube apparatus can be endoscopically placed during the initial placement procedure for the apparatus. Presented now, is an embodiment of a method for endoscopically placing the PEG-J apparatus 100 disclosed and discussed above.

The tool engagable structure 140 of the PEG-J tube unit 105 is configured for allowing placement using the well-known Ponsky-pull technique. Using such technique, an endoscope is introduced down the upper airway through a patient's esophageus into the stomach while the patient is under conscious sedation. Using the endoscope, the stomach in insufflated with air to its maximum diameter and the left upper quadrant of the abdominal wall is palpated or balloted. Good tenting of the gastric lumen 185 should be identified endoscopically as well as transillumination of the abdominal wall to prevent placing the PEG-J tube unit 105 through the colon or other intraabdominal organs. The abdomen is then prepped and draped, and local anesthesia is infiltrated over the selected sight.

A small incision is now made over the skin with a scalpel. A sheath (i.e., tube) is introduced through the incision from the abdominal wall of the patient into the gastric lumen 185 under endoscopic visualization. A first end portion of a double looped wire (i.e., a pull-through device) is introduced through the sheath into the gastric lumen 185. The wire is then grasped with a snare or other suitable tool through the endoscope, followed by the endoscope being withdrawn along with the wire being pulled through the esophageus and out the mouth as the endoscope is withdrawn. The length of the wire is such that it extends through the incision in the abdominal wall, through the gastric lumen 185, through the esophageus and out of the patient's mouth.

With an end portion of the wire exposed through the patient's mouth, the tool engagable structure 140 of the PEG-J tube unit 105 is attached to the first end portion of the wire (i.e., the oral end portion of the wire). The end portion of the wire exposed through the abdominal wall is pulled, thereby pulling the PEG-J tube unit through the mouth, esophageus and the stomach. The PEG-J tube unit 105 is pulled until the posterior bumper 130 is firmly engaged against the gastric mucosa of the stomach with the interconnected tubes 120, 125 extending through the stomach and abdominal walls (See FIG. 1). The anterior bumper 110 is then fed over the interconnected tubes 120, 125 from the abdominal side and pushed down to the skin to hold the stomach and abdominal wall together. The interconnected tubes 120, 125 are then severed for detaching the tool engagable structure 140 from the interconnected tubes 120, 125 and exposing the internal passages of the interconnected tubes 120, 125. Optionally, the end portions of the interconnected tubes 120, 125 exposed at the abdominal wall are then separated by cutting and/or peeling them apart. Such separation terminates at or before the anterior bumper 110.

To prevent air from leaking out the G-tube 120 as the stomach is reinsufflated with air, a valve body is engaged into the passage of the G-tube 120 tube at the separated end portion. The J-tube introducer port 125 is the cut to a desired length. Thereafter, the J-tube 115 is fed down into the passage 125′ of the J-Tube introducer 125 far enough to prevent leakage of air on subsequent reinsufflation of the stomach. In the case where the plug 155 is fixed with respect to the elongated portion 165 of the J-tube 115, the length of the J-tube introducer 125 influences how far from the incision the tip region 170 of the J-tube 115 can be placed downstream of the stomach. This is important because, jointly, the length of the elongated portion 170 of the J-tube and the length of the J-tube introducer 125 must accommodate the tip region 170 of the J-tube 115 being placed through the pyloric valve of the patient. For patients having different physical and physiological attributes, cutting the J-tube introducer 125 to length allows for adjustment of the as-installed position of the tip region of the J-tube.

Now, the endoscope is reintroduced into the upper airway and through the esophageus into the stomach. The gastric lumen 185 is reinsufflated with air. The posterior bumper 130 is examined for bleeding and good approximation. The tip region 170 of the J-tube 115 is then endoscopically fed (i.e., placed) down through the pyloric valve into the duodenum as far distally as possible and the endoscope is withdrawn back into the gastric lumen 185. Such placing of the tip region 170 of the J-tube 115 is performed after or in concert with inserting the J-tube into the passage 125′ of the J-tube introducer 125. The reason for this insertion requirement is so as to limit an amount of the J-tube 115 disposed within the stomach prior to the tip region 170 (i.e., first end portion) of the J-tube 115 being placed through the pyloric valve. It is undesirable for the J-tube 115 to become coiled within the gastric lumen 185.

The stomach is then desufflated (i.e., excess air therein is removed) and the endoscope is withdrawn. The J-tube 115 is further inserted into the J-tube introducer 125 until the plug 155 of the J-tube 115 is securely engaged within the passage of the J-tube introducer 125. The seal 162 is then secured onto the J-tube introducer 125. Preferably, but not necessarily, the elongated portion 165 of the J-tube 115 includes at least about 2-3 inches of excess length (i.e., slack portion of the J-tube 115 disposed within the gastric lumen 185) when the plug 155 of the J-tube 115 is securely engaged within the passage of the J-tube introducer 125.

At this point the PEG-J apparatus can be used in a conventional manner. One such use is providing tube feeds via the J-tube 115. Another such use is performing aspiration of the stomach by exposing the G-tube 120 to suction. The tip region 170 of the J-tube 115 will continue to migrate through the duodenum into the jejunum as the bowel peristalses (i.e., pushes) it through distally.

One problem with J-tubes is that they tend to clog because of their small diameter. Over time, tube feeds delivered via the J-tube begin to build up in the J-tube and it becomes clogged. This has been a discouraging aspect of other types of G-J tube apparatuses. With conventional J-tube and G-J tube apparatuses, once the J-tube clogs off, the whole apparatus must be removed and the tip region (i.e., jejunal region) of a replacement J-tube tube has to be placed back into the duodenum under endoscopic or fluoroscopic (x-ray) guidance. This is because the tip region of the J-tube does not pass through the pyloric valve into the duodenum on its own with every tube change (e.g., due to coiling of the J-tube).

In accordance with the present invention, the joint functionality of the J-tube introducer and J-tube advantageously allows the J-tube to be changed without having to remove or replace the whole G-J tube apparatus. Furthermore, such replacement of the J-tube in accordance with the present invention advantageously does not require placement of the J-tube through the pyloric valve with either endoscopic or fluoroscopic guidance. In one embodiment of the present invention, an elongated sheath is used for enabling a J-tube configured and endoscopically placed in accordance with the present invention to be changed without having to remove or replace the whole G-J tube apparatus and without having to perform placement of the J-tube through the pyloric valve with either endoscopic or fluoroscopic guidance.

To this end, any means for securing the seal 162 is removed, thereby allowing the J-tube 115 to be suitably pulled back (e.g., about 2-3 inches) from the J-tube introducer 125. Accordingly, the plug 155 and seal 162 become separated from the J-tube introducer 125. Next, the J-tube 115 is severed at a location between the J-tube introducer 125 and the plug 155. A guide member is inserted into the passage of the J-tube 115. The guide member has an outside diameter less than or about equal to that of the J-tube 115. A sheath is now fed over the guide member and advanced over the J-tube 115 through the passage 125′ of the J-tube introducer 125. Thus, it is required that the sheath has an inside diameter about the same as or slightly larger than an outside diameter of the J-tube 115 and has an outside diameter about the same as or slightly smaller than an inside diameter of the passage 125′ of the J-tube introducer 125. The J-tube 115 remains in place until sheath is suitably advanced over the J-tube 115 (i.e., its fully advanced position). For example, by means such as length comparison of the J-tube 115 and the sheath, the sheath is fed over the J-tube until the sheath is known to have passed through the pyloric valve and/or past the tip region 170 of the J-tube 115. When the sheath is in such a fully advanced position, the previously installed J-tube 115 is now fully retracted from within the sheath and a replacement J-tube, which is configured in accordance with the present invention (e.g., identical to the previously installed J-tube), is inserted into the sheath to the same or similar distance as the previously installed J-tube. The upper end of the sheath is cut broken apart, broken away or extracted, thereby allowing the plug 155 of the replacement J-plug to be properly inserted into the passage 125′ of the J-tube introducer and allowing a seal 162 to be installed and secured in place.

In the preceding detailed description, reference has been made to the accompanying drawings that form a part hereof, and in which are shown by way of illustration specific embodiments in which the present invention may be practiced. These embodiments, and certain variants thereof, have been described in sufficient detail to enable those skilled in the art to practice embodiments of the present invention. It is to be understood that other suitable embodiments may be utilized and that logical, mechanical, chemical and electrical changes may be made without departing from the spirit or scope of such inventive disclosures. To avoid unnecessary detail, the description omits certain information known to those skilled in the art. The preceding detailed description is, therefore, not intended to be limited to the specific forms set forth herein, but on the contrary, it is intended to cover such alternatives, modifications, and equivalents, as can be reasonably included within the spirit and scope of the appended claims.

What is claimed is:

1. A method for providing a jejunostomy tube and a gastrostomy tube within a body of a patient during a single endoscopic procedure, comprising:
endoscopically placing a gastrostomy-jejunostomy (G-J) tube apparatus through abdominal and stomach walls of the patient such that a first end portion of the G-J tube apparatus is exposed within the gastric lumen of the stomach and a second end portion of the G-J tube apparatus is exposed outside of said patient's body, wherein the G-J tube apparatus includes provisions configured for compressing together portions of said walls encompassing the G-J tube apparatus, wherein the G-J tube apparatus includes two interconnected tubes extending in a side-by-side orientation with respect to each other and wherein a first one of said interconnected tubes is configured for functioning as a gastrostomy tube (G-tube) and a second one of said interconnected tubes is configured for functioning as a jejunostomy tube (J-tube) introducer;
inserting a first end portion of a J-tube into a passage of the J-tube introducer from an end portion thereof located outside of the patient's body, wherein said inserting is performed in a manner that causes the first end portion of the J-tube to become exposed within the gastric lumen of the stomach; and
endoscopically placing the first end portion of the J-tube through a pyloric valve of the patient, wherein said placing is performed one of after and in concert with said inserting.

2. The method of claim 1 wherein endoscopically placing the first end portion of the J-tube is performed in concert with said inserting so as to limit an amount of the J-tube disposed within the stomach prior to the first end portion of the J-tube being placed through the pyloric valve.

3. The method of claim 2, further comprising:
providing a fluid-resistant seal within the passage of the J-tube introducer between the J-tube introducer and the J-tube after completing said placing of the first end portion of the J-tube, wherein the seal provides a fluid-resistant seal between the J-tube introducer and the J-tube.

4. The method of claim 3, wherein:
the seal is integral with the J-tube; and
providing the seal includes continuing said inserting at least until the seal first becomes engaged within the passage of the J-tube introducer.

5. The method of claim 4, further comprising:
partially retracting the J-tube from within the J-tube introducer to a position whereby the seal becomes is fully retracted from within the sheath;
severing the j-tube at a location between the seal and the J-tube introducer thereby removing the seal from the J-tube;
slideably inserting a first end portion of a sheath into the passage of the J-tube introducer over the J-tube, wherein the sheath is slideably inserted until the sheath extends through the pyloric valve;
fully retracting the J-tube from within the sheath such that the J-tube is no longer within the sheath; and
inserting a first end portion of a new J-tube into the sheath and through the J-tube introducer until the first end portion of the J-tube extends beyond the first end portion of the sheath.

6. The method of claim 3, further comprising:
separating said interconnected tubes from each other along the second end portion of the G-J tube apparatus, wherein said interconnected tubes are separated from each other over a prescribed distance starting from an end face of the G-J tube apparatus at the second end portion thereof.

7. The method of claim 6, further comprising:
removing a portion of the second one of said interconnected tubes at the separated end thereof after performing said separating and prior to performing said inserting, wherein the J-tube includes a plug portion integral with an exterior surface thereof adjacent an end portion thereof, wherein providing said seal includes continuing said inserting at least until the plug portion first becomes engaged within the passage of the J-tube introducer whereby performing said removing at least partially influences a minimum length of the J-tube inserted through the J-tube introducer when the plug portion first becomes engaged within the passage of the J-tube introducer.

8. The method of claim 1 wherein endoscopically placing the first end portion of the J-tube is performed in concert with said inserting so as to limit an amount of the J-tube disposed within the stomach prior to the first end portion of the J-tube being placed through the pyloric valve.

9. A percutaneous endoscopic gastrostomy-jejunostomy (PEG-J) tube apparatus, comprising:
a percutaneous endoscopic gastrostomy-jejunostomy (PEG-J) tube unit including two interconnected tubes extending in a side-by-side orientation with respect to each other and a posterior bumper encompassing both of said tubes adjacent a first end portion thereof, wherein each one of said interconnected tubes includes a passage extending therethrough between opposing end faces thereof and wherein a first one of said interconnected tubes is configured for functioning as a gastrostomy tube (G-tube) and a second one of said interconnected tubes is configured for functioning as a jejunostomy tube (J-tube) introducer; and
a J-tube slideably inserted into a passage of the J-tube introducer, wherein the J-tube includes a plug portion integral therewith and disposed within the passage of the J-tube introducer for providing a fluid-resistant seal between the J-tube and the J-tube introducer.

10. The apparatus of claim 9 wherein said interconnected tubes are connected to each other in a manner allowing said interconnected tubes to be manually peeled apart from each other generally along a prescribed interface.

11. The apparatus of claim 10 wherein the portion of the J-tube slideably inserted into the passage of the J-tube introducer has a substantially uniform outside dimension.

12. The apparatus of claim 11, further comprising:
an anterior bumper slideably engaged over said interconnected tubes, wherein the PEG-J tube unit includes a posterior bumper integral with an end portion thereof and wherein the anterior bumper is slideable to within a prescribed distance from the posterior bumper.

13. The apparatus of claim 9 wherein the portion of the J-tube slideably inserted into the passage of the J-tube introducer has a substantially uniform outside dimension.

14. A system of components configured for performing placement and maintenance of a percutaneous endoscopic gastrostomy-jejunostomy (PEG-J) tube apparatus, comprising:
a percutaneous endoscopic gastrostomy-jejunostomy (PEG-J) tube unit including two interconnected tubes extending in a side-by-side orientation with respect to each other and a posterior bumper encompassing both of said tubes adjacent a first end portion of the PEG-J tube unit, wherein a first one of said interconnected tubes is configured for functioning as a gastrostomy tube (G-tube) and a second one of said interconnected tubes is configured for functioning as a jejunostomy tube (J-tube) introducer;
an anterior bumper having a passage extending through a central portion thereof, wherein the passage is configured for having said interconnected tubes extending therethrough; and
a jujenostomy tube (J-tube) including an elongated portion configured for being slideably inserted through a passage of the J-tube introducer and a plug portion attached to the elongated portion thereof, wherein the plug portion is configured for being engaged within a passage of the J-tube introducer for providing a fluid-resistant seal between the J-tube and the J-tube introducer.

15. The system of claim 14 wherein the elongated portion of the J-tube has a substantially uniform outside dimension that allows an entire length of the elongated portion to be slideably inserted into the passage of the J-tube introducer.

16. The system of claim 15 wherein:
the passage of the J-tube introducer has a substantially uniform inside dimension; and
said inside and outside dimensions are jointly configured for allowing a tubular sheath to be inserted over the J-tube and through the passage of the J-tube introducer when the elongated portion of the J-tube is extending through the passage of the J-tube introducer.

17. The system of claim 14 wherein said interconnected tubes are connected to each other in a manner allowing said interconnected tubes to be manually peeled apart from each other generally along a prescribed interface.

18. The system of claim 14 wherein:
the PEG-J tube unit further includes an tool engagable structure integral with a second end portion thereof; and
each one of said interconnected tubes includes a passage that extends through a face of a first end thereof and are terminated by the tool engagable structure.

19. The system of claim 14 wherein:
the elongated portion of the J-tube has a substantially uniform outside dimension that allows an entire length of the elongated portion to be slideably inserted into the passage of the J-tube introducer;
the passage of the J-tube introducer has a substantially uniform inside dimension;
said inside and outside dimensions are jointly configured for allowing a tubular sheath to be inserted over the J-tube and through the passage of the J-tube introducer when the elongated portion of the J-tube is extending through the passage of the J-tube introducer; and
said interconnected tubes are connected to each other in a manner allowing said interconnected tubes to be manually peeled apart from each other generally along a prescribed interface.

20. The system of claim 19 wherein:
the PEG-J tube unit further includes an tool engagable structure integral with a second end portion thereof; and
each one of said interconnected tubes includes a passage that extends through a face of a first end thereof and are terminated by the tool engagable structure.
